(19) United States
(12) Patent Application Publication
(10) Pub. No.: US 2012/0226122 A1
(43) Pub. Date: Sep. 6, 2012

(54) Inserter for in-vitro analyte sensor

(76) Inventors: Arturo Meuniot, San Francisco, CA (US); Julie Dahlgard, Miami, FL (US); Andrew Lewis Johnston, Redwood City, CA (US)

(21) Appl. No.: 13/041,074

(22) Filed: Mar. 4, 2011

(51) Int. Cl. A61B 5/145

(52) U.S. Cl. 600/365; 600/309

(57) ABSTRACT

A device and method for implanting an analyte sensor into a subcutaneous fat layer is presented. The device comprises a housing that is positioned above the subcutaneous fat layer, a blade shuttle, and a sensor shuttle. In one embodiment, a spring is compressed between the blade shuttle and the sensor shuttle. The blade shuttle and sensor shuttle move towards the subcutaneous fat layer. When a spring force is released by the spring, the blade shuttle moves towards and pierces into the subcutaneous fat layer creating a pathway into the subcutaneous fat layer. The analyte sensor is implanted by the sensor shuttle by following the blade shuttle into the pathway created by the blade shuttle. The blade shuttle is then retracted from the subcutaneous fat layer, leaving the analyte sensor in the fat layer.

### BACKGROUND

The present disclosure generally relates to a device and method for inserting an implantable analyte sensor for a continuous monitoring system into a subcutaneous fat layer and, in particular, relates to a device and method for inserting an implantable analyte sensor for a continuous monitoring system into a subcutaneous fat layer using a staged insertion of a flat needle, followed by the analyte sensor.

A continuous monitoring system for the monitoring an analyte, such as, for example, glucose, can record analyte levels in a fat layer constantly throughout the day and night. One advantage of such a continuous monitoring system is its ability to identify changes and trends in analyte levels that can not easily be detected with intermittent standard tests, such as, for example, in the case of glucose, HbAlc levels or random finger stick measurements.

Continuous monitoring systems typically use a tiny implantable sensor that is inserted under the skin, or into the subcutaneous fat layer, typically in the abdomen area, to check an analyte levels in the tissue fluid. The sensor can stay in place for several days to a week and then is replaced. A transmitter sends information about the analyte levels via, for example, a wire to a monitor or wirelessly by radio waves from the sensor to a wireless monitor. The user can check blood samples with a traditional glucose meter in order to program the sensor. The systems can provide substantially real-time measurements of analyte levels, with analyte levels typically being displayed at one to five minute intervals.

Users can set alarms to alert them when analyte levels are too high or too low. Special software is available to download data from the continuous monitoring systems to, for example, a computer, for tracking and analysis of patterns and trends, and the continuous monitoring systems can also display trend graphs on a display. The device can capture data points related hypoglycemic events, especially those overnight, hyperglycemic events, such as those between meals, early morning spikes, to help evaluate how diet and exercise affect analyte levels, and can provide several days review of the effects of changes made during therapy by the user's health care professional team.

Therefore, there is a need for an as small as possible device and method to insert the analyte sensor for a continuous monitoring system into the subcutaneous fat layer as easily, quickly, seamlessly, painlessly and as cost effectively as possible.

### SUMMARY

According to the present disclosure, a device for implanting an analyte sensor into a subcutaneous fat layer is disclosed. The device can comprise a housing that is positioned above the subcutaneous fat layer, a blade shuttle, and a sensor shuttle. The blade shuttle can comprise a piercing needle at a first end of the blade shuttle closest to the subcutaneous fat layer. The analyte sensor can be positioned at a first end of the sensor shuttle closest to the subcutaneous fat layer.

In accordance with one embodiment of the present disclosure, the blade shuttle and the sensor shuttle can move along cam paths in the housing resulting in the blade shuttle and sensor having arced trajectories.

In accordance with another embodiment of the present disclosure, the device can provide for a staged insertion of the implantable analyte sensor, wherein the staged insertion can comprise an insertion of the piercing needle on the blade shuttle first, followed by the implantable analyte sensor on the sensor shuttle.

In accordance with still another embodiment of the present disclosure, the blade shuttle and the sensor shuttle can be hinged together at a rotable point that can rotate the shuttles towards the skin. The blade shuttle and the sensor shuttle can also be hinged on the same axis within the housing.

In accordance with yet another embodiment of the present disclosure, the implanting device can be stored flat, positioned on the body of the patient and then un-folded into a substantially “L” shape. An internal drive spring can be primed during the unfolding operation and can subsequently be triggered in order to allow for the vertical insertion of an analyte sensor into the subcutaneous fat layer of the patient. The device can then fold flat for minimum size after insertion.

Accordingly, it is a feature of the embodiments of the present disclosure to provide as small as possible device and method for the insertion of the analyte sensor for a continuous monitoring system into the subcutaneous fat layer as easily, quickly, seamlessly, painlessly and as cost effectively possible. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates an inserter of an analyte sensor for a continuous monitoring system according to an embodiment of the present disclosure.

FIG. 2 illustrates an exploded view of the inserter of an analyte sensor for a continuous monitoring system shown in FIG. 1 according to an embodiment of the present disclosure.

FIG. 3 illustrates an inserter of an analyte sensor for a continuous monitoring system according to another embodiment of the present disclosure.

FIG. 4 illustrates an exploded view of the inserter of an analyte sensor for a continuous monitoring system shown in FIG. 3 according to an embodiment of the present disclosure.

FIGS. 5 illustrates an inserter of an analyte sensor for a continuous monitoring system according to still another embodiment according to an embodiment of the present disclosure.

FIG. 6 illustrates an exploded view of the inserter of an analyte sensor for a continuous monitoring system shown in FIG. 5 according to an embodiment of the present disclosure.

FIG. 7 illustrates an exploded view of the inserter of an analyte sensor for a continuous monitoring system shown in FIG. 5 according to another embodiment of the present disclosure.

FIGS. 8 illustrates another inserter of an analyte sensor for a continuous monitoring system according to still another embodiment according to an embodiment of the present disclosure.

FIG. 9 illustrates an inserter of an analyte sensor for a continuous monitoring system according to yet another embodiment of the present disclosure.

FIG. 10 illustrates an exploded view of the inserter of an analyte sensor for a continuous monitoring system shown in FIG. 9 according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

A device for inserting and implanting an analyte sensor for a continuous monitoring system that is small as possible, that has a low manufacturing cost and that is easy to use is presented. In general, this device can be disposable and can be wearable for at least three days. The device can have an integrated piercing and insertion mechanism for the placement of an implantable sensor into the subcutaneous fat layer of the user. It can be advantageous to have the piercing mechanism hidden within the device to avoid as much as possible the unease of needle-phobic users, for example. In one embodiment, the piercing mechanism can pierce to a depth of approximately 2 mm to approximately 7 mm. In another embodiment, the piercing mechanism can pierce to a depth of approximately 3 mm to approximately 5 mm. In still another embodiment, the piercing mechanism can pierce to a depth of approximately 5 mm. In one embodiment, the insertion mechanism can insert the implantable sensor to a depth of approximately 6 mm to approximately 12 mm. In another embodiment, the insertion mechanism can insert the implantable sensor to a depth of approximately 8 mm to approximately 10 mm. In still another embodiment, the insertion mechanism can insert the implantable sensor to a depth of approximately 8 mm.

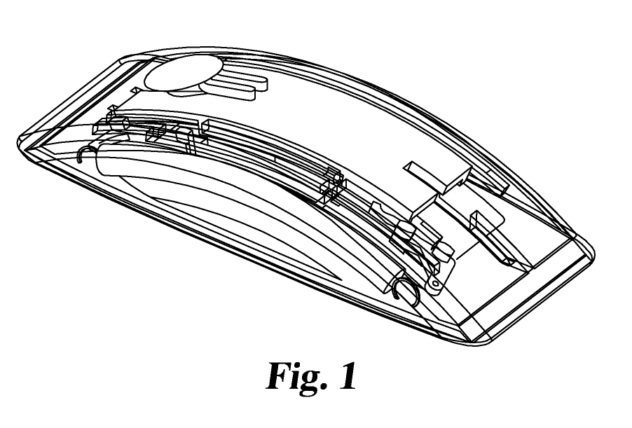

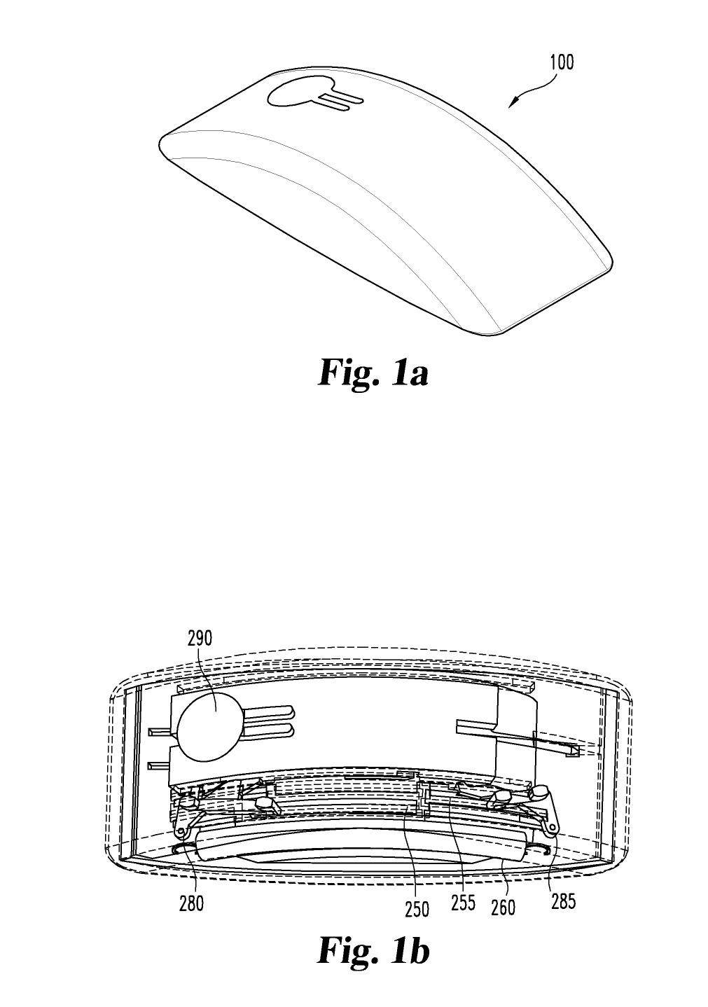

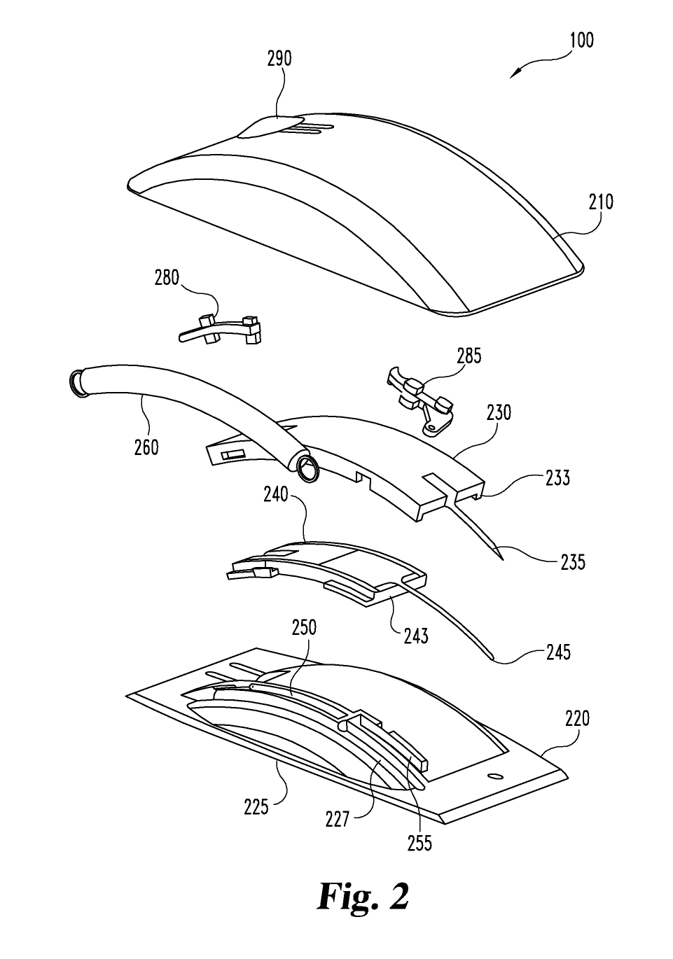

Referring initially to FIGS. 1 and 2, one embodiment of a device 100 for inserting and implanting an analyte sensor for a continuous monitoring system is disclosed. This device 100 can achieve the three motions (i.e., piercing, analyte sensor insertion and needle retraction) needed to implant an analyte sensor 245 with a single spring 260. In one example embodiment, the implantable analyte sensor 245 can sense an analyte concentration in-vitro. In one example embodiment, the implantable analyte sensor 245 can detect glucose concentration in a subcutaneous fat layer of a patient. In one example embodiment, the implantable analyte sensor 245 can be a “needle-type” sensor. However, any other suitable implantable analyte sensors known in the art can be used. In one example embodiment, the single spring 260 can drive all three motions of the device 100 with, for example, a preload force of about 5.5 Newtons; a piercing force of about 3.9 Newtons, and a retraction force of about 0.4 Newtons. The timing of the device 100 can be internally controlled and can be fully automatic through the use of this spring 260, as will be discussed below. In one example embodiment, the device 100 can have an overall length of approximately 50 mm to approximately 55 mm, an overall height of approximately 7 mm to approximately 10 mm, and an overall width of approximately 22 mm to approximately 27 mm.

In one embodiment, the device 100 can comprise a upper housing 210 and a lower housing 220. In one example embodiment, the lower housing 220 can be attached to the skin of the patient using known attachment methods. In one embodiment, the lower housing 220 can be attached to the skin of the patient, such as, for example, around the abdomen region. However, any other suitable placement of the lower housing 220 to the patient will not deviate from the spirit of the present disclosure. In one embodiment, the upper housing 210 can be affixed onto the lower housing 220 in order to provide a thinner profile against the body of the patient. In one embodiment, the lower housing 220 can be affixed to the upper housing 210 by ultrasonic welding. In another embodiment, the lower housing 220 can be affixed by gluing the lower housing 220 to the upper housing 210. However, any suitable method of affixing known in the art can be used.

Turning to FIG. 2, in one embodiment, the device 100 can utilize a needle 235 and an implantable analyte sensor 245. In one embodiment, the needle 235 and the implantable sensor 245 can be mounted on a first end 233 of a blade shuttle 230 and a first end 243 of a sensor shuttle 240, respectively. In one embodiment, the needle 235 can be substantially flat. In another embodiment, the needle 235 can be substantially arced convexly. In one embodiment, the needle 235 can be formed in sheet metal and bowed convexly. In one embodiment, the implantable analyte sensor 245 can also be bowed convexly.

In one embodiment, the blade shuttle 230 and the sensor shuttle 240 can slide on curved guides in the lower housing 220 in a substantially arced trajectory. In this embodiment, the curved shape of the blade shuttle 230 and the sensor shuttle 240 can match their arced trajectory. In one embodiment, the sensor shuttle 240 of the device 100 can be positioned over a substantially convex protrusion 227 on upper surface of the lower housing 220 formed by a lower recess 225 in a bottom surface of the lower housing 220. A blade shuttle 230 can be positioned on top of the sensor shuttle 240. In one embodiment, the blade shuttle 230 can be substantially arced convexly. In one embodiment, the blade shuttle 230 can be oriented horizontally. In one embodiment, the sensor shuttle 240 can have a substantially arced, or convex, shape. By arcing the sensor shuttle 240 and blade shuttle 230, the total overall dimensions of the device 100 can be minimized. In one embodiment, the sensor shuttle 240 and blade shuttle 230 can be arced over a power supply, such as, for example, a battery, which can also minimize the overall footprint of the device 100.

On one embodiment, the spring 260 can be then positioned into the device 100. In one embodiment, the spring 260 can be positioned to fully extend between a fore shuttle 285 and a rear shuttle 280. The spring 260 can always be under tension. Therefore, the spring 260 will be trying to pull the rear shuttle 280 forward and the fore shuttle 285 rearward. The rear shuttle 280 can be engaged with the blade shuttle 230 and the sensor shuttle 240. The fore shuttle 285 can initially be engaged with the lower housing 220 and/or upper housing 210 in such a manner that the fore shuttle 285 cannot move. After the spring 260 is positioned, the upper housing 210 can be placed on top of the lower housing 220 and affixed. In one example embodiment, the upper housing 210 can be affixed to the lower housing 220 as described above. In one embodiment, the blade shuttle 230 can initially be engaged with the upper housing 210 so that the blade shuttle 230 does not slide forward despite the fact that the rear shuttle 280 will be trying to pull the blade shuttle 230 forward due to the tension of the spring 260. Having the blade shuttle 230 initially engaged with the upper housing also can prevent the rear shuttle 280 from pulling the sensor shuttle 240 forward.

In one example embodiment, the upper housing 210 can have a curvature that substantially matches that of the lower housing 220. In one embodiment, the upper housing 220 can have a curved surface that is approximately 9 mm at its highest point. In one embodiment, a trigger 290 can be incorporated into the upper housing 220. In one embodiment, the trigger 290 can be covered by an elastomer overmold. However, any other suitable coverings can be used. In one embodiment, after assembly, the upper housing 210 and the lower housing 220 can then be fully sealed, making the device 100 substantially waterproof.

In one embodiment, the blade shuttle 230 and sensor shuttle 240 can be spring-loaded by the spring 260 as described above and can be activated by the patient by the trigger 290, for example, to insert the needle 235 into the skin by moving the blade shuttle 230 through its curved trajectory. In one embodiment, the blade shuttle 230 can have initial entry angle of about 30°. In one embodiment, the needle 235 can initially be latched into the retracted position. The blade shuttle 230 can be activated to move towards the subcutaneous fat layer by a patient action such as, for example, on a manual (or automated) trigger 290. After the patient activates the trigger 290, the blade shuttle 230 disengages from the upper housing 210.

In one embodiment, the activated trigger 290 can initiate movement of the rear shuttle 280 along a track 250 in the substantially convex protrusion 227 in the lower housing 220 due to the disengagement of the blade shuttle 230 from the upper housing 210. This movement can result in the release the pre-loaded spring 260 causing the blade shuttle 230 to move forward to insert the needle 235 into the subcutaneous fat layer. Once the blade shuttle 230 reaches its full depth, the rear shuttle 230 can disengage the blade shuttle 230 after hitting a stop in the track 250. As the blade shuttle 230 is driven to its full depth in the subcutaneous fat layer, the spring 260 can simultaneously drive the sensor shuttle 240 and the implantable analyte sensor 245 subcutaneously through the hole made by the needle 235 into the subcutaneous fat layer by sliding the sensor shuttle 240 along the same arced trajectory. In one embodiment, the needle 235 can make contact with the subcutaneous fat layer first because the needle 235 can be started further forward (i.e., closer to the subcutaneous fat layer) than the implantable analyte sensor 245.

When the analyte sensor 245 is at its full, operational depth, the rear shuttle 280 can halt its forward progress by reaching the end of its track 250 and can disengage from the blade shuttle 230. At this point, the fore shuttle 285 can disengage from the lower housing 220 and/or upper housing 210 and can engage the blade shuttle 230. Then, the spring force of the spring 260 can cause the fore shuttle 285 to move along the track 255 of the fore shuttle 285 in the substantially convex protrusion 227 in the lower housing 220; thereby, pulling the blade shuttle 230 rearward away from the subcutaneous fat layer. In other words, the needle 235 and the blade shuttle 230 can be returned to its original position within the device 100 by retraction force of the spring 260. Thereby, the rear shuttle 280 and the fore shuttle 285 can act as the timing mechanism for the device 100 based on their location within their tracks 250, 255. In one embodiment, the rear shuttle 230 can remain engaged with sensor shuttle 240 so that rear shuttle 280 can hold the analyte sensor 245 forward as the needle 235 is retracted. After the retraction of the blade shuttle 230, the implanted analyte sensor 245 can remain in the subcutaneous fat layer. In one embodiment, the blade shuttle 230 does not begin to retract until analyte sensor 245 is fully implanted.

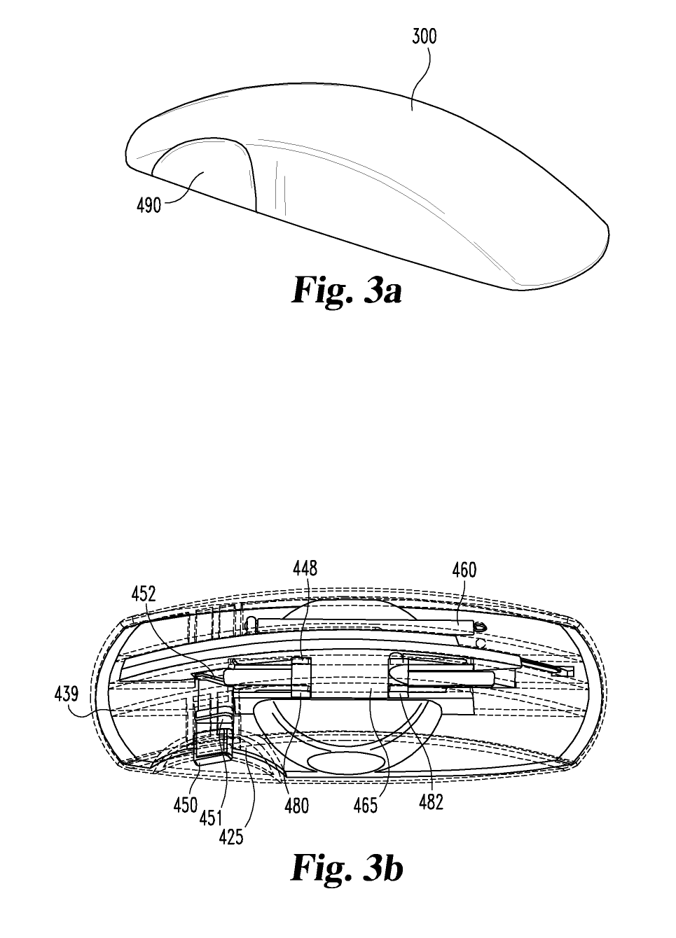

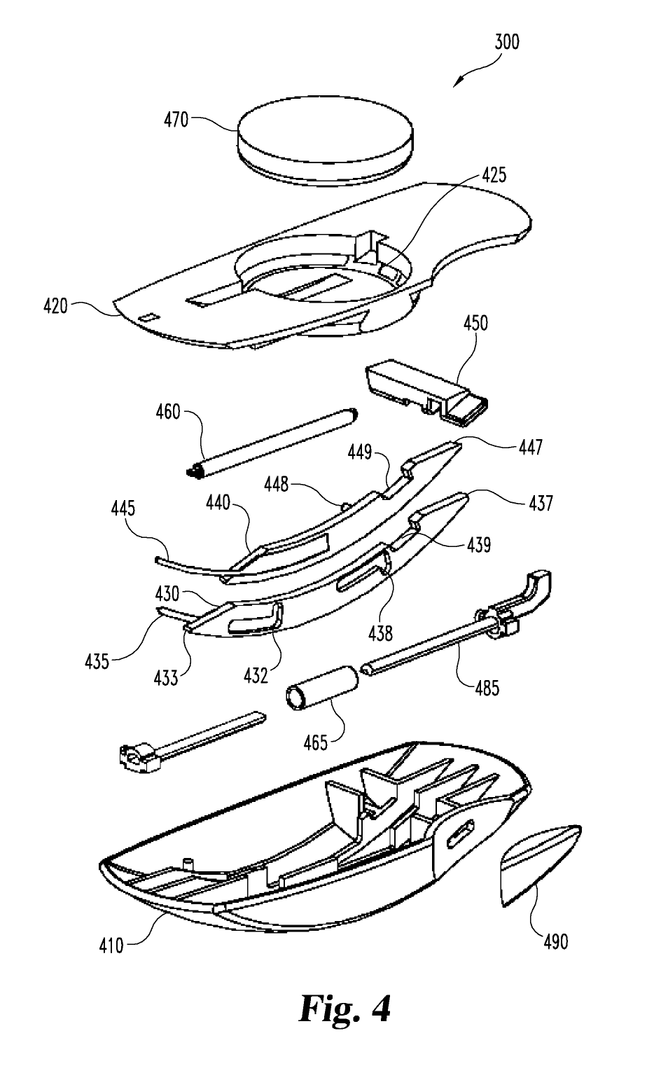

Turning to FIGS. 3 and 4, another embodiment of the device 300 is disclosed. This device 100 can achieve the piercing and needle retraction with a single compression spring 460 and the analyte sensor insertion with a single extension spring 465. The timing of the device 300 can be internally controlled by an internal slider 450 that can be advanced across the housing 410, 420 of the device 300 and can fully be automatic by the use of these springs 460, 465, as will be discussed below. In one example embodiment, the device 300 can have an overall length of about 55 mm to about 60 mm, an overall height of about 8 mm to about 10 mm, and an overall width of about 22 mm to about 25 mm.

In one example embodiment, this device 300 can comprise an upper housing 410 and a lower housing 420. In one example embodiment, the upper housing 410 can be have a substantially convex shape. In one example embodiment, the upper housing 410 can be affixed to the lower housing 420 in order to provide a thinner profile against the body of the patient. In one example embodiment, the lower housing 420 can be affixed to the upper housing 410 by ultrasonic welding. In another example embodiment, the lower housing 420 can be affixed by gluing the lower housing 420 to the upper housing 410. However, any suitable method of affixing known in the art can be used. As discussed above, in one embodiment, after being affixed, the upper housing 410 and the lower housing 420 can then be fully sealed, making the device 300 substantially waterproof. In one example embodiment, the lower housing 420 can then be attached to the skin of the patient around the abdomen region, for example. However, any other suitable placement of the lower housing 420 to the patient will not deviate from the spirit of the present disclosure.

In one example embodiment, the device 300 can comprise a blade shuttle 430 and a sensor shuttle 440. In one embodiment, the sensor shuttle 440 can comprise an implantable analyte sensor 445 positioned at a first end 443 of the sensor shuttle 440. In one example embodiment, the implantable analyte sensor 445 can sense an analyte concentration in-vitro. In one example embodiment, the implantable analyte sensor 445 can detect glucose concentration in a subcutaneous fat layer of a patient. However, any other suitable implantable analyte sensors known in the art can be used. In one example embodiment, the implantable analyte sensor 445 can be a “needle-type” sensor. However, any other suitable implantable analyte sensors known in the art can be used. In one embodiment, the implantable analyte sensor 445 can be substantially flat and can be cut into a substantially arc shape. In this embodiment, the implantable analyte sensor 445 is not bent or bowed.

In one embodiment, the blade shuttle 430 can comprise a needle 435 that can be positioned at a first end 433 of the blade shuttle 430. In one embodiment, the needle 435 can be substantially flat. However, any other suitable needles known in the art can be used. In one embodiment, the needle 435 can be formed from sheet metal that can then be cut into a substantially arc shape. In this embodiment, the sheet metal of the needle 435 is not bent or bowed.

In one embodiment, the blade shuttle 430 and the sensor shuttle 440 can be substantially curved with a substantially convex shape similar to the substantially convex shape of the upper housing 410. In one embodiment, the upper housing 410 can have a curved surface that is about 9 mm at its highest point. In one embodiment, the sensor shuttle 440 and the blade shuttle 430 can be oriented perpendicular to the skin of the patient so that a simple two-dimensional outline can be used to arc the first end 433 of the blade shuttle 430 and the first end 443 of the sensor shuttle 440 down to the skin without having to pre-form the needle 435 or bend the sensor 445. In one embodiment, the sensor shuttle 440 and blade shuttle 430 can travel over a power supply, such as, for example, a battery 470, which can also minimize the footprint of the device 300.

The blade shuttle 430 and the sensor shuttle 440 can be positioned within the upper housing 410. The device 300 can also comprise a rear shuttle 480 and a fore shuttle 482. In one embodiment, the compression spring 460 can be used to drive the rear shuttle 480 and the fore shuttle 482 apart. In one embodiment, the fore shuttle 482 can initially engage the blade shuttle 430. In one embodiment, the slider 450 can be positioned within the upper housing 410 in order to engage both the blade shuttle 430 and the sensor shuttle 440. In one embodiment, the fore shuttle 482 can exert force to try and push the blade shuttle 430 forward. However, the blade shuttle 430 will be held in place (i.e., not moved) by the slider 450. In an example embodiment, an extension spring 460 can engage the sensor shuttle 440 and can be stretched the length of the upper housing 410. In one embodiment, the extension spring 460 can try to exert a force and pull the sensor shuttle 440 forward but the sensor shuttle 440 can also be held in place by the slider 450. Because the slider 450 can only move side-to-side through the device 300, the slider 450 can effectively hold both the compression spring 460 and the extension spring 465 in their energized states since this side-to-side movement of the slider 450 is orthogonal to the spring forces, which are directed front-to-back.

In one example embodiment, a battery 470 can be affixed into a recess 425 located in the lower housing 420 in order to provide a source of power to the device 300. The battery 470 can then be covered with a label using any method known in the art.

In one example embodiment, a trigger 490, such as, for example, a button, switch or any other suitable method of triggering known in the art, can be affixed to the upper housing 420. In one embodiment, the trigger 490 can be an elastomer area on the side of the upper housing 420 that can be actuated by the patient pressing or squeezing this elastomer area. Once the trigger 490 is actuated, the slider 450 can be automatically advanced across the device 300 resulting in the sequential release of the compression spring 465 and the extension spring 460 as well as in the movement of the blade shuttle 430, the sensor shuttle 440 and the forward and rear shuttles 480, 482. In other words, the trigger 490 can be used by the patient to activate, or trigger, the movement of the slider 450 in order to place the implantable analyte sensor 445 into the subcutaneous fat layer of the patient.

In one embodiment, the slider 450 can comprise a series of cams. The series of cams can comprise, for example, a ramp cam 451 and a stop cam 452 to engage the blade shuttle 430 and the sensor shuttle 440. In one exemplary embodiment, the slider 450 can move substantially perpendicular to the lower housing 420 and the subcutaneous fat layer of the patient. At the same time the slider 450 is moving substantially perpendicular, the series of slider cams 451, 452 can engage the blade shuttle 430 and sensor shuttle 440.

In one embodiment, as the slider 450 moves into the upper housing 410, the blade shuttle 430 can be released. The fore shuttle 482 can then begin pushing the blade shuttle 430 forward along its track resulting in the needle 435 of the blade shuttle 430 initially piercing the subcutaneous fat layer. In one embodiment, the blade shuttle 430 can have initial entry angle of approximately 35°. As the blade shuttle 430 advances, the blade shuttle 430 can engage the ramp cam 451 of the slider 450 which can drive the slider 450 further into the device 300. As the fore shuttle 482 drives the blade shuttle forward, the fore shuttle 482 can gradually disengage from the blade shuttle 430. The fore shuttle 482 can completely disengage from the blade shuttle 430 when the blade shuttle 430 comes in contact with the recess 425 of the lower housing 420, thereby stopping forward progress of the blade shuttle 430. As the blade shuttle 430 completes its travel forward, the blade shuttle 430 can come in contact with and can engage the rear shuttle 480. The rear shuttle 480 can now be ready to retract the needle 435. However, the slider 450, at this point, can still be holding the rear shuttle 480 forward.

After the blade shuttle 430 has been fully advanced, the blade shuttle 430 has moved the slider 450 far enough into the interior of the device 300 that the slider 450 can release the sensor shuttle 440. At this point, the extension spring 465 can then pull the sensor shuttle 440 along its track in order the insert the implanted analyte sensor 445 into the subcutaneous fat layer. As the sensor shuttle 440 moves forward, the sensor shuttle 440 can then engage the ramp cam 451 of the slider 450 to continue to drive the slider 450 even further into the device 300. When the sensor shuttle 440 is fully advanced, the sensor shuttle 440 has moved the slider 450 far enough into the device 300 such that the slider 450 can then release the rear shuttle 480. The rear shuttle 480 can then drive the blade shuttle 430 rearward, thereby, removing the needle 435 from the subcutaneous fat layer. After the retraction of the blade shuttle 430, the implantable analyte sensor 445 can remain in the subcutaneous fat layer.

By using the two springs 460, 465, a staged insertion of the blade shuttle 430, followed by the analyte sensor 445 implantation, and then the removal of the blade shuttle 430 can be possible. This staged insertion can be less painful to the patient. However, an additional spring is needed to achieve the staged insertion.

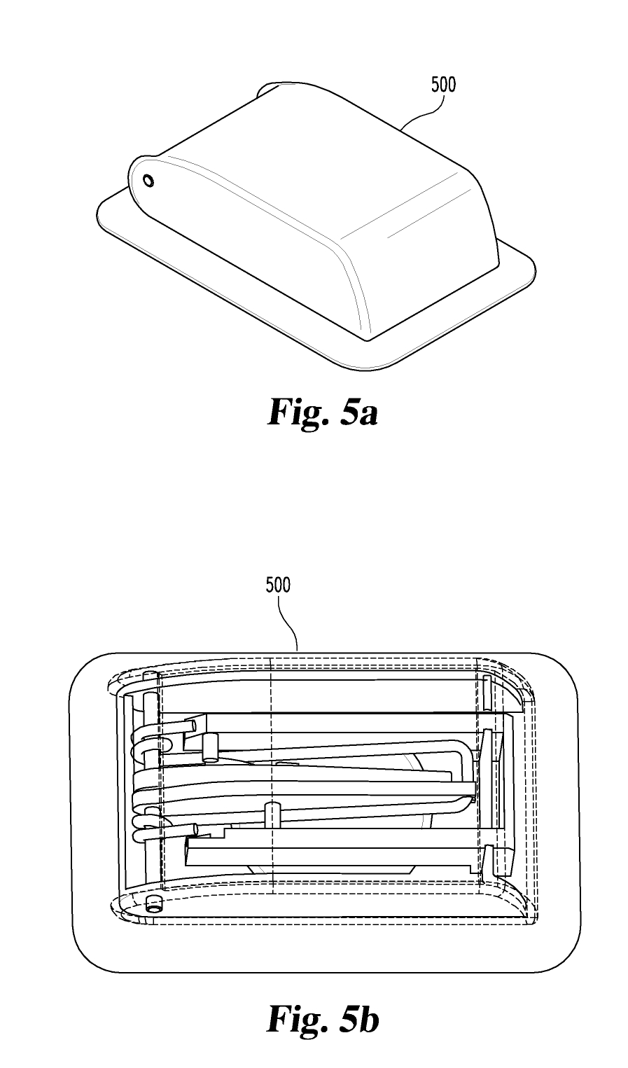

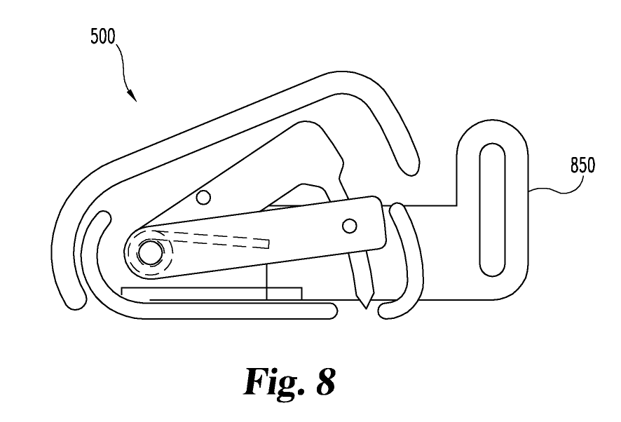

Another exemplary embodiment of an device 500 comprising an upper housing 610 and a lower housing 620 is shown in FIGS. 5-7. In one embodiment, an adhesive pad 605 can be used to adhere the device 500 to the user's skin. In one embodiment, a slider 650 can have a cam profile embedded on either side of the slider 650, i.e., a blade shuttle 630 cam profile 653 and a sensor shuttle 640 cam profile 654. In one embodiment, the slider 650 can be substantially “U” shape and can be slid into the lower housing 620. In another embodiment, as illustrated in FIG. 8, a substantially flat slider 850 can have a cam profile embedded on either side of the slider 850, i.e., a blade shuttle 630 cam profile and a sensor shuttle 640 cam profile. In this embodiment, the slider 850 can be pulled from the device 500 and discarded.

Once the slider 650 is slid into the device 500, or once the slider 850 is removed from the device 500, the upper housing 610 collapses onto the lower housing 620, thereby reducing the overall size of the device 500. In other words, the device 500 utilizes this collapsing housing design to minimize unused space within the device 500, which can reduce the final volume of the device 500 worn by the user.

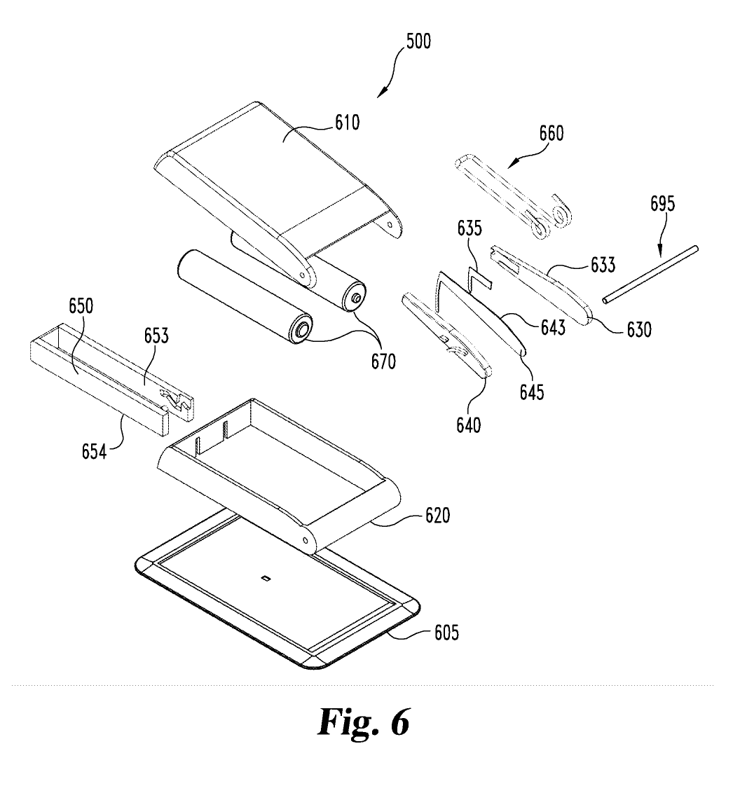

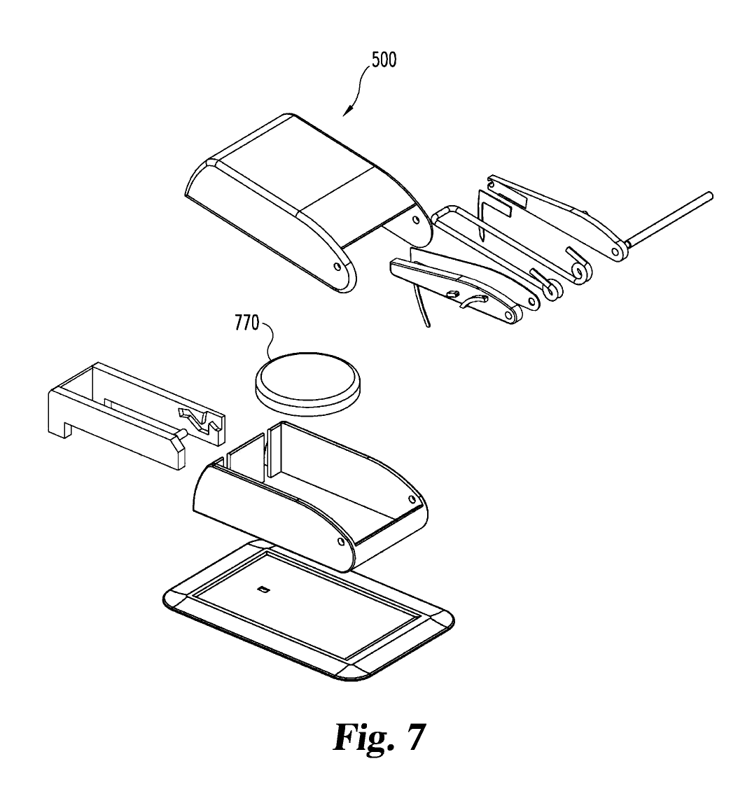

Turning to FIGS. 6 and 7, a needle 635 can be attached to a first end 633 of a blade shuttle 630 and an implantable analyte sensor 645 can be attached to a first end 643 of a sensor shuttle 640 in a manner similar to that described above. In one example embodiment, the implantable analyte sensor 645 can sense an analyte concentration in-vitro. In one example embodiment, the implantable analyte sensor 645 can detect glucose concentration in a subcutaneous fat layer of a patient. In one example embodiment, the implantable analyte sensor 645 can be a “needle-type” sensor. However, any other suitable implantable analyte sensors known in the art can be used. In one embodiment, the needle 635 can be substantially flat. However, any other suitable needles known in the art can be used.

A pin 695 can run along the width of the device 500 to hold the blade shuttle 630, sensor shuttle 640, spring 660, upper housing 610 and lower housing 620 together. In one example embodiment, the blade shuttle 630 and sensor shuttle 640 can be pinched between spring loops 660 to maintain the position of the blade shuttle 630 and sensor shuttle 640 within the device 500. The blade shuttle 630 and the sensor shuttle 640 can rotate about the pin 695. In one embodiment, the device 500 can be shipped with the spring 660 in a lower stress state by utilizing the cam profiles on the slider 650.

In one embodiment, the sensor shuttle 340 and the blade shuttle 130 can be oriented perpendicular to the skin of the patient so that a simple two-dimensional outline can be used to arc the first end 643 of the sensor shuttle 640 and the first end 633 of the blade shuttle 630 down to the skin without having to pre-form the needle 635 or bend the implantable analyte sensor 645. In one example embodiment, a battery 770 as shown in FIG. 7, or batteries 670 as shown in FIG. 6, can be placed into position on the lower housing 620 as a source of power. In FIG. 6, in one example embodiment, the batteries 670 can be at least two AAA batteries. In FIG. 7, in one example embodiment, the battery 770 can be a coin cell battery.

When the patient moves the slider 650 into, or out of, the lower housing 620, the cam profile embedded on blade shuttle 630 side 653 of the slider 650 can engage the blade shuttle 630 and can initially rotate the blade shuttle 630 on the pin 695 away from the subcutaneous fat layer, thereby, energizing the spring 660. The blade shuttle 630, as it follows the cam profile on the blade shuttle 630 side 653 of the slider 650, can then be quickly forced into the subcutaneous fat layer by the spring force of the spring 660. In one example embodiment, the sensor shuttle 640 can engage the cam profile embedded on the sensor shuttle 640 side 654 of the slider 650. By following the sensor shuttle cam profile 654, the sensor shuttle 640 can then follow the blade shuttle 630 and implant the implantable sensor 645. Upon actuation by the patient by the movement of the slider 650 into, or out of, the device 500, the spring 660 can be first wound and then can be released to have the blade shuttle 630 automatically pierce the skin. In one embodiment, the blade shuttle 630 cam profile 653 on the slider 650 coupled with the sprung blade shuttle 630 can create substantially instant piercing but slower (i.e., patient-controlled) sensor 645 insertion.

Alternatively, the piercing of the skin by the blade shuttle 630 can also be user-controlled. In this embodiment, the blade shuttle 630 cam profile 653 embedded on the slider 650 would not have the blade shuttle 630 initially pull back from the subcutaneous fat layer but, instead, would directly pierce the skin as the patient pushes the slider 650 into, or out of, the device 500. One possible benefit of this approach can be that spring is not necessary for operation.

In one embodiment, the independent cam profiles on opposite sides of the slider 650, i.e., the blade shuttle 630 cam profile side 653 and the sensor shuttle 640 cam profile side 654, can control the timing of the blade shuttle 630 and the sensor shuttle 640 independently. Timing can be easily adjustable by modifying the cam profiles on the sides 653, 654 of the slider 650. Further, gradual velocity changes can be possible by varying the cam profiles. It can also be appreciated that the velocity changes can also be dependent upon whether the blade shuttle 630 and sensor shuttle 640 are being spring or user-controlled. The simple pivot rotation of the device 500 can be reliable and can be easily manufactured.

In the example embodiment illustrated in FIG. 6, i.e., the embodiment comprising two or more batteries, the upper housing 610 can have a length of about 55 mm to about 60 mm, a height after insertion of about 8 mm to about 12 mm, and a width of about 20 mm to about 35 mm. In one example embodiment, the overall device 500 can have a length of about 65 mm to about 70 mm, and a width of about 40 mm to about 45 mm. The greater overall size can be due to the additional of the adhesive pad 605. In one embodiment, the device 500 can have an insertion angle between about 95° to about 100°.

In the example embodiment illustrated in FIG. 7, i.e., the embodiment comprising a single battery, the upper housing 310 can have a length of about 40 mm to about 45 mm, a height after insertion of about 10 mm to about 15 mm, and a width of about 22 mm to about 26 mm In one example embodiment, the overall device 500 can have a length of about 45 mm to about 42 mm, and a width of about 20 mm to about 35 mm. The greater overall size can be due to the additional of the adhesive pad 605. In one embodiment, the device 500 can have an insertion angle between about 100° to about 105°.

As can be seen, the device 500 in FIG. 6 can have a larger configuration than the device 500 in FIG. 7, mainly, due to the size of the batteries used in the device 500. However, the device 500 having at least two batteries 670, in FIG. 6, has the potential of storing more electrical energy than the device 500 having a single battery 770 of FIG. 7. The additional electrical energy may be an advantage depending on the electronics needed in the device 500.

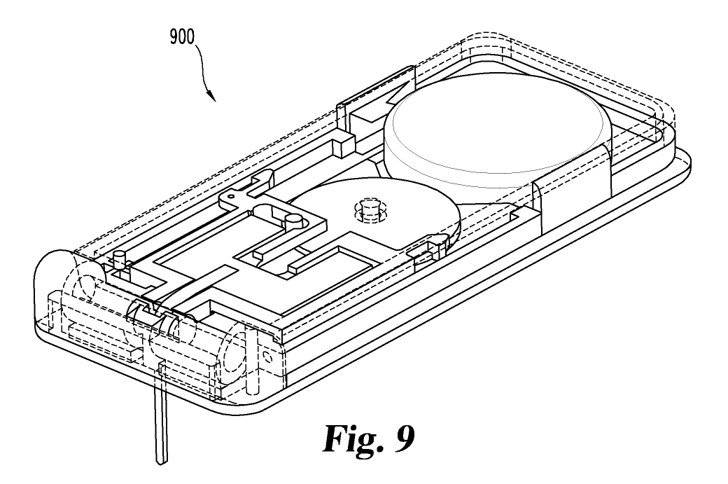

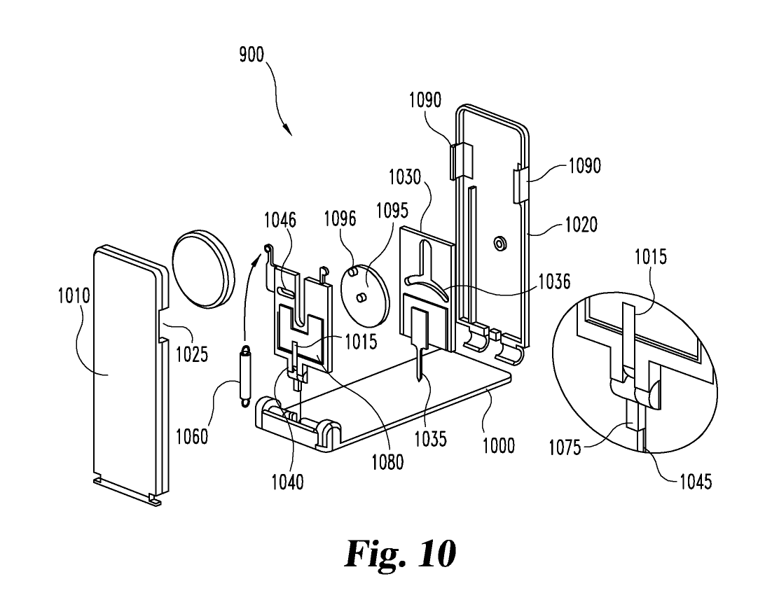

In yet another exemplary embodiment, illustrated in FIGS. 9 and 10, an inserting device 900 can have a housing comprising a base plate 1000, a upper housing 1010 and a lower housing 1030. In one embodiment, the device 900 can have an overall length of about 60 mm, an overall height of about 7 mm, and an overall width of about 25 mm. This device 900 can achieve the three motions (i.e., piercing, analyte sensor insertion and needle retraction) needed to implant the analyte sensor 1045 with a rotatable cam 1095 and a single extension spring 1060.

In one embodiment, the blade shuttle 1030, a sensor shuttle 1040 and a rotatable cam 1095, engaged with and positioned between the blade shuttle 1030 and the sensor shuttle 1040, can be inserted into a lower housing 1020. In one embodiment, the cam 1095 can be a quarter wheel mechanism. The cam 1095 can have a sensor shuttle pin 1096 that engages a slot 1046 on the sensor shuttle 1040. The cam 1095 can also have a blade sensor pin (not shown) on the opposite side of the cam 1095. The blade sensor pin engages a slot 1036 on the blade shuttle 1030. An extension spring 1060 can be connected to the sensor shuttle 1040 and lower housing 1020. In one embodiment, the extension spring 1060 can always exert a force and pull on the sensor shuttle 1040. A battery 1070 can be inserted into a recess 1025 in the bottom of the upper housing 1010. The upper housing 1010 can be affixed to the lower housing 1020 by any suitable method known in the art as was discussed above regarding the other embodiments.

In one example embodiment, on one end of the blade shuttle 1030 there can be a blade, or needle 1035. In one embodiment, the sensor shuttle 1040 can comprise an implantable analyte sensor 1045 and an electronics component 1080 such as, for example, a printed circuit board (PCB). In one embodiment, the electronics component 1080 can be connected to the implantable analyte sensor 1045 via a flex cable 1015. The electronics component 1080 can contain circuitry to control the implantable analyte sensor 1045 and can be powered by the battery 1070. In one example embodiment, during shipment or storage, the upper housing 1010 and the lower housing 1020, and all of the various components of the inserting device 900 therein described above can be assembled into each other against the base plate 1000, making a small, substantially flat package. In this embodiment, for example, both the blade shuttle 1030 and the sensor shuttle 1040 can be retracted into the inserting device 900 during shipment or storage.

In one example embodiment, the implantable analyte sensor 1045 can sense an analyte concentration in-vitro. In one example embodiment, the implantable analyte sensor 1045 can detect glucose concentration in a subcutaneous fat layer of a patient. In one example embodiment, the implantable analyte sensor 1045 can be a “needle-type” sensor. However, any other suitable implantable analyte sensors known in the art can be used.

In one example embodiment, the base plate 1000 of the inserting device 900 can be adhered to the skin by the patient above the subcutaneous fat layer. In one embodiment, the base plate 1000 can be attached, for example, to the abdomen region of the patient. In one example embodiment, to begin insertion, the upper housing 1010 and the lower housing 1020 can be rotated together upward away from the base plate 1000 and away from the body of the patient into a substantially vertical orientation to the skin, at approximately 90 degrees. In this orientation, the upper housing 1010 and lower housing 1020 can form a substantially “L” shape with the base plate 1000. In one example embodiment, the vertical rotation upwards of the upper housing 1010 and lower housing 1020 can prime the extension spring 1060. In another example embodiment, the extension spring 1060 can be manually primed by the patient. In yet another example embodiment, the extension spring 1060 can be shipped in the prime state. In one embodiment, the vertical rotation upwards of the upper housing 1010 and lower housing 1020 can place the single extension spring 1060 under tension. In this embodiment, the extension spring 1060 can be attempting to exert a force and pull the sensor shuttle 1040 forward. However, the sensor shuttle 1040 can be latched to the upper housing 1010 and/or the lower housing 1020 causing the extension spring 1060 to be held in tension and the sensor shuttle 1040 not to move.

In one example embodiment, firing buttons 1090, or triggers, can be pressed, or squeezed, by the patient, in order to release the sensor shuttle 1040 from the upper housing 1010 and/or the lower housing 1020 to drive the cam 1095. In one embodiment, at least two firing buttons 1090 can be molded integrally with the lower housing 1020 on opposite sides of the lower housing 1020. In one embodiment, the firing buttons 1090 can be molded flexible tabs, for example. In one example embodiment, after both of the at least two firing buttons 1090 are pressed, the firing buttons 1090 can flex inward and can disengage the sensor shuttle 1040 from upper housing 1010 and/or the lower housing 1020, thereby, allowing the extension spring 1060 to pull the sensor shuttle 1040 forward (i.e., downwards towards the subcutaneous fat layer). By having at least two firing buttons 1090 that both need to be pressed to actuate the device 900, the chances of an accidental triggering of the device 900 can be reduced. As the sensor shuttle 1040 moves forward, the cam 1095 can rotate.

In one embodiment, as the sensor shuttle 1040 is being pulled forward by the extension spring 1060, the sensor shuttle pin 1096 can engage with the slot 1036 causing the cam 1095 to rotate. As the cam 1095 rotates, the blade shuttle pin can move along the slot 1036 so that the blade shuttle 1030 can move forward to first pierce the skin. As the cam 1095 continues to rotate, the blade shuttle pin can continue to move along the slot 1036, thereby, withdrawing the blade shuttle 1030 from the subcutaneous fat layer. As the cam 1095 rotates, the sensor shuttle 1030 is still being driven downwards towards the skin of the patient by the extension spring 1060 allowing the implantable analyte sensor 1045 to follow the pathway into the subcutaneous fat layer created by the blade 1035 of the blade shuttle 1030. In one embodiment, the spring 1060 can continue to hold the sensor shuttle 1040 biased forward against a hard stop, such as, for example, the base plate 1000, thereby, leaving the implantable analyte sensor 1045 in the subcutaneous fat layer.

In other words, because the blade shuttle 1030 can be out-of-phase with respect to the sensor shuttle 1040 (via the cam 1095), the blade shuttle 1030 can accelerate forward, stop and then retract rearward while the sensor shuttle 1030 can be moving forward the entire time. This out-of-phase movement and the relative starting positions of the blade shuttle 1030 and sensor shuttle 1040 can also mean that the needle 1035 can pierce the subcutaneous fat layer before the tip of the implantable analyte sensor 1045 arrives at the incision site. As the sensor shuttle 1030 moves downward, the extension spring 1060 can compress resulting in the implantable analyte sensor 1045 remaining in the subcutaneous fat layer.

In one example embodiment, the upper housing 1010 and lower housing 1020 can be then folded back onto the base plate 1000 to create a substantially flat inserting device 900. In one embodiment, the cam 1095 can automatically ramp the blade shuttle 1030 and the sensor shuttle 1040 velocities up and down more smoothly due to the sinusoidal characteristics of rotary motion through the use of the slot 1036 in the blade shuttle 1030 and the slot 1046 in the sensor shuttle 1040. This smooth movement can be in contrast to the use of instant releases and hard stops. This smooth movement in turn can also reduce shock loading throughout the device 900 which, in turn, can reduce the discomfort of the patient.

In one example embodiment, a sensor mount 1075 in the sensor shuttle 1030 can dock into the base plate 1000 and can support the implantable analyte sensor 1045 as the upper housing 1020 and lower housing 1010 are rotated, or folded, down into place onto the base plate 1000. By the sensor mount 1075 of the implantable analyte sensor 1045 docking in the base plate 1000 and the flex circuit 1015 making the connection with the electronics component 1080, the implantable analyte sensor 1045 substrate bending can be essentially eliminated when the device 900 is folded flat against the patient's body. By actuating a vertical orientation that can then be folded down for ongoing wear can result in a device 900 that can have a minimal device thickness because all actuation can occur in a flat plane while oriented vertically. Additionally, by driving the blade shuttle 1030 and sensor shuttle 1040 in substantially 90 degree orientation, the device 900 can require minimal blade 1035 and sensor 1045 tip lengths because the tips can take a relatively short path to their desired location.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

For the purposes of describing and defining the present disclosure, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A device for implanting an analyte sensor into a subcutaneous fat layer, the device comprising:
a housing positioned above the subcutaneous fat layer;
a blade shuttle comprising a first end, wherein the blade shuttle comprises a piercing needle at the first end of the blade shuttle; and
a sensor shuttle comprising a first end, wherein the analyte sensor is positioned at the first end of the sensor shuttle;
wherein the first end of blade shuttle rotates towards and pierces the subcutaneous fat layer with the piercing needle to create an incision and wherein the first end of the sensor shuttle follows the first end of the blade shuttle into the incision implanting the analyte sensor in the subcutaneous fat layer.

2. The device of claim 1, wherein the housing is substantially convex shaped.

3. The device of claim 2, wherein the blade shuttle and the sensor shuttle are substantially curved in a convex shaped.

4. The device of claim 3, wherein the curved blade shuttle and the curved sensor shuttle move along curved grooves in the substantially convex housing resulting in the curved blade shuttle and curved sensor having arced trajectories.

5. The device of claim 3, further comprising,
a trigger for activated the rotation of the curved blade shuttle and the curved sensor shuttle by releasing the stored energy of the spring.

6. The device of claim 1, wherein stored energy of the spring results in the retraction of the blade shuttle from the subcutaneous fat layer after the implantation of the analyte sensor.

7. The device of claim 1, wherein the piercing needle is substantially flat.

8. The device of claim 1, further comprising,
a battery for powering the device.

9. The device of claim 1, wherein the spring is extended between a fore shuttle and a rear shuttle.

10. The device of claim 1, further comprising,
a cam.

11. The device of claim 1, further comprising,
a slider that rotates the blade shuttle to energize the spring.

12. The device of claim 1, further comprising,
a slider that moves through the device.

13. The device of claim 12, wherein the slider has embedded cam profiles to control the blade shuttle and the sensor shuttle.

14. The device of claim 12, wherein the slider is removed from the device to trigger the actuation of the device.

15. The device of claim 1, wherein the housing comprises an upper housing and a lower housing, wherein the upper housing is affixed to the lower housing and wherein the lower housing is attached to skin above the subcutaneous fat layer.

16. The device of claim 1, wherein the device is substantially water-proof.

17. The device of claim 1, wherein the device can be used with a continuous monitoring system.

18. The device of claim 1, wherein the analyte sensor senses glucose concentration.

19. A device for implanting an analyte sensor into a subcutaneous fat layer, the device comprising:
a substantially flat housing comprised of a upper housing, a lower housing and a base plate, wherein the substantially flat housing is positioned above the subcutaneous fat layer;
a blade shuttle positioned within the lower housing, wherein the blade shuttle comprises a needle at one end of the blade shuttle; and
a sensor shuttle positioned within the lower housing, wherein the analyte sensor is positioned at one end of the sensor shuttle; and
a rotatable cam positioned between and engaged with the blade shuttle and the sensor shuttle;
wherein the rotation of the cam results in the needle of the blade shuttle piercing the subcutaneous fat layer to create an incision, followed by the sensor shuttle implanting the analyte sensor in the incision the subcutaneous fat layer.

20. The device for claim 19, wherein when the substantially flat housing is in a substantially vertical position, the substantially flat housing forms a substantially “L” shape with the base plate.

21. The device for claim 19, wherein the rotatable cam continues to engage the blade shuttle resulting in the blade shuttle being retracted from the subcutaneous fat layer.

22. The device of claim 19, further comprising,
a battery for powering the device positioned in the upper housing.

23. The device of claim 19, further comprising,
a flex cable for connecting the analyte sensor to an electronics component.

24. A method for implanting an analyte sensor into a subcutaneous fat layer, the method comprising:
positioning a blade shuttle and a sensor shuttle within a housing and wherein the blade shuttle and sensor shuttle move towards the subcutaneous fat layer;
releasing the blade shuttle so that the blade shuttle rotates towards and pierces the subcutaneous fat layer, creating a pathway into the subcutaneous fat layer; and
implanting the analyte sensor by a sensor shuttle that follows the pathway of blade shuttle into the subcutaneous fat layer.

25. The method of claim 24, wherein after implantation of the analyte sensor, the blade shuttle rotates out of the subcutaneous fat layer.
